(19) United States
(12) Patent Application Publication
(10) Pub. No.: US 2011/0217251 A1
(43) Pub. Date: Sep. 8, 2011

(54) THERAPEUTIC COMPOSITIONS COMPRISING POLYHYDROXYLTATE FATTY ALCOHOL DERIVATIVES AND USES THEREOF

(76) Inventors: Shai Meretzki, Haifa (IL); Gennady Rosenblat, Haifa (IL); Joseph Segal, Haifa (IL)

(21) Appl. No.: 13/062,758

(22) PCT Filed: Sep. 8, 2009
(86) PCT No.: PCT/IL2009/000875

§ 371 Date: May 30, 2011

Related U.S. Application Data
(60) Provisional application No. 61/136,468, filed on Sep. 8, 2008.

(51) Int. Cl. A61K 8/34; A61K 31/22; A61K 31/047; A61K 8/37; A61Q 17/04; A61P 17/16; A61P 9/10; A61P 1/00; A61P 19/02

(52) U.S. Cl. 424/59; 514/546; 514/738

(57) ABSTRACT

Therapeutic compositions comprising pharmaceutically effective amounts of isolated polyhydroxylated fatty alcohols, in particular suitable for topical or systemic administration. A method for the isolation of a natural polyhydroxylated fatty alcohol from a fruit or vegetable source is also disclosed.

### FIELD OF THE INVENTION

The present invention relates to therapeutic compositions and uses thereof, and more specifically to a therapeutic composition comprising polyhydroxylated fatty alcohols or acetyl derivatives thereof for the prevention and treatment of the disorders are associated with increased T-cell proliferation and abnormal expression of TNF-alpha, IFN γ and phospholipase A2 activity.

### BACKGROUND OF THE INVENTION

T lymphocytes are common participants in the inflammatory response associated with various form of tissue injury. The inflammation process is characterized by T lymphocyte migration into the inflamed tissue; these lymphocytes were found to be responsible for many inflammatory reactions and are an important contributor to these reactions.

It is well established that environmental damage including UV cause a large number of major disturbance to cells of the immune system including activation of T and B cells. Immuno-histochemical studies have identified infiltrating the lymphoid cells in damaged and sun-exposed tissues. Residual epidermal T-lymphocytes activated by the environmental damage and UV exposure thought to be the initial source of INF-alpha and IFN-γ. The environmental damage and the exposure to ultraviolet (UV) irradiation has been found to cause induction of several inflammatory-associated enzymes such as phospholipase A2 and cyclooxygenase-2 (COX-2), which are believed to be responsible with pro-inflammatory mediators such as interferon (IFN) gamma and tumor necrosis factor (TNF)-alpha, for acute skin inflammation and subacute chronic inflammation.

IFN gamma and TNF alpha are two important pro-inflammatory cytokines, which the T lymphocytes contribute to the inflammatory reaction.

Tumor necrosis factor (TNF) is a cell-associated cytokine that is processed from a 26 kd precursor to a 17 kd active form. TNF has been shown to be a primary mediator of inflammation, fever, and acute phase responses of several diseases in humans and animals.

Recent study have shown that extensive production of TNF alpha induces onset of variety of disease, including cachexia attributed to cancer or infectious diseases (Beutler B, Greenwald D, Hulmes J D, Chang M, Pan Y.-C. E. Mathison J, Ulevitch R., Cerami A. Nature (London). 1985;316:552-4), septic shock (Starnes H F, Jr, Pearce M K, Tewari A., Yim J H, Zou J.-C, Abrams J. S. “Anti-IL-6 monoclonal antibodies protect against lethal Escherichia coli infection and lethal tumor necrosis factor-a challenge in mice”. J. Immunol. 1990;145:4185-91; Beutler, B., Milsark, I. W., Cerami, A. C. “Passive Immunization Against Cachectin/Tumor Necrosis Factor Protects Mice from Lethal Effect of Endotoxin”, Science. 1985; 229:869-71); chronic rheumatoid arthritis (Tetta C, Camussi G, Modena V, Vittorio C Di, Baglioni C. “Tumor necrosis factor in serum and synovial fluid of patients with active and severe rheumatoid arthritis”. Ann. Reum. Dis. 1990; 49:665-7; Elliott M J, Maini R N, Feldmann M, Long-Fox A, Charles P, Bijl H, Woody J N, “Repeated therapy with monoclonal antibody to tumor necrosis factor alpha (cA2) in patients with rheumatoid arthritis”. Lancet. 1994; 344: 1125-7), inflammatory disease such as ulcerative colitis and Crohn disease (MacDonald T T, Hutchings P, Choy M-Y, Murch S, Cooke A. “Tumor necrosis factor alpha and interferon-gamma production measured at the single cell level in normal and inflamed human intestine”. Clin Exp Immunol. 1990;81:301-5), osteoarthritis (Venn G, Nietfeld J J, Duits A J, Brennan F M, Arner E, Covington M, Billingham M E, Hardingham T E. “Elevated synovial fluid levels of interleukin-6 and tumor necrosis factor associated with early experimental canine osteoarthritis.” Arthritis Reum. 1993; 36: 819-26), Kawasaki's disease (Matsubara T, Furukawa S, Yabuta K, “Serum levels of tumor necrosis factor, interleukin 2 receptor, and interferon-gamma in Kawasaki disease involved coronary-artery lesions”. Clin. Immunol. Immunopathol. 1990; 56:29-36), multiple sclerosis (Sharief M K, Hentges R. “Association between tumor necrosis factor-alpha and disease progression in patients with multiple sclerosis”. N. Engl. J. Med. 1991; 325 (7):467-72), type II diabetics (Hotamisligil G S, Shargill N S, Spiegelman B M. “Adipose expression of tumor necrosis factor-alpha: direct role in obesity-linked insulin resistance”, Science. 1993; 259: 87-91) mycobacterium infection, psoriasis and other inflammatory skin disease.

TNF-alpha is also considered to be one of the most important tissue factors involved in the epidermal damage in response to chronic and acute solar radiation, and has been implicated in early stage skin carcinogenesis. TNF alpha and TNF alpha type 1 receptor knockout mice have been shown to be protected against squamous cell carcinoma (Arnott C H, Scott K A, Moore R J, Robinson S C, Thompson R G, Balkwill F R. “Expression of both TNF-alpha receptor subtypes is essential for optimal skin tumor development”, Oncogene. 2004; 23(10):1902-10).

TNF-alpha is produced by a wide variety of cells, including macrophages, natural killer cells, T lymphocytes, and keratinocytes. Different stimuli appear to induce TNF-alpha production via different regulatory mechanisms and TNF alpha mediate disorders via different targets molecules.

The effect of TNF alpha on tissue remodeling may be synergistically potentiated by interferon gamma (IFN)-γ. IFN gamma is another cell factor which has been implicated in the development of different disorders and inflammatory syndrome, including for example UV associated skin cancer. Experimental data has suggested a synergistic effect of TNF alpha and IFN gamma in epithelial cell function and the significance of their role in the pathology of inflammatory bowel disease (Ito R, Shin-Ya M, Kishida T, Urano A, Takada R, Sakagami J, Imanishi J, Kita M, Ueda Y, Iwakura Y, Kataoka K, Okanoue T, Mazda O, “Interferon-gamma is causatively involved in experimental inflammatory bowel disease in mice”. Clin Exp Immunol. 2006; 146(2):330-8). In addition, IFN-γ synergistically potentiate TNF alpha induced activity of nuclear factor (NF) kappa B (Cheshire J L, Baldwin A S, JR. “Synergistic activation of NF-kB by tumor necrosis factor alpha and gamma Interferon via enhanced IkBa degradation and de novo IkBb degradation”. Mol. Cell. Biol. 1997; 6746-54). Similarly, to TNF alpha, INF γ may be also produced in responds to environmental damage and solar radiation. A small but significant and sustained production of IFN gamma in epidermal skin exposed to UV irradiation was previously reported by Shen et al. (Shen J, Bao S, Reeve V E: “Modulation of IL-10, IL-12, and IFN-gamma in the epidermis of hairless mice by UVA (320-400 nm) and UVB (280-320 nm) radiation”. J Invest Dermatol. 1999; 113:1059-64).

But for inducing expression of inflammatory cytokines like TNF alpha, INF γ, Exposure to environmental damage and to UV radiation also were demonstrated to causes significant activation of the enzyme phospholipase A2 in the skin and other damaged tissues (Greshma A, Masferrer J, Chen X, Leal-Knouri S, Pentland A. “Increased synthesis of high-molecular weight cPLA₂ mediates early UV-induced PGE₂ in human skin”, Am. J. Physiol., Cell Physiol. 1996; 39(4): C1037-C1050). Phospholipase A2 (PLA2) is a family of enzymes that catalyze the liberation of Arachidonic Acid (AA) from positions 2 of the cellular phospholipids and initiate a complex cascade of biochemical reaction that lead to the synthesis of eicasonoids. The AA can be metabolized through the lipoxygenase or cyclooxygenase pathways, and form leucotrienes, prostaglandins and thromboxanes, all of which are bioactive lipids involved in tissue damage and inducing inflammatory reaction (Serhan C N, Haegstromm J Z, Leslie C C. “Lipid mediator networks in cell signaling: update and impact of cytokines”, FASEB J. 1996; 10: 1147-58).

A number of endogenous messengers and environmental factors other than solar radiation provoke dramatic activation of PLA2 followed by AA liberation and transformation into inflammatory intermediates. Activation of PLA2 and the liberation of AA are closely associated with another enzyme, cyclooxygenase (COX)-2 that catalyzes the first step in the conversion of arachidonic acid into eicosanoids and has a critical role in carcinogenesis (Fischer S M, Pavone A, Mikulec C, Langenbach R, Rundhaug J E. “Cyclooxygenase-2 expression is critical for chronic UV-induced murine skin carcinogenesis”, Mol Carcinog. 2007; 46(5): 363-71)

Multiple studies have demonstrated that PGE2 mediates signals is involved in the induction of inflammation, angiogenesis, vasodilatation, and vascular permeability. This PGE2 signaling pathway promotes the development of carcinogenesis. Moreover, endogenous enzymatic oxidation of AA by COX or lipoxygenase has long been recognized as a contributing factor in the development of various types of cancer. Recent experiments have demonstrated that nuclear oxygenase activity may result in the co-oxidation of DNA via AA free radical intermediates, and that AA-peroxidation can efficiently induce mutations in mammalian cells and promote DNA stand breaks. These increased level of AA may exert genotoxic effects and elevate cancer risk.

Due to the fact that T cell proliferation and infiltration of inflammatory induced cells to the damaged tissue are the major activator of the inflammatory process, T cells inhibitors were considered to use to delay this process. Currently, a number of T lymphocyte inhibitors, PLA2 activity inhibitors, TNF-alpha, and IFN-γ expression inhibitors, are known which are useful for the treatment of various pathological conditions. Among these are powerful anti-inflammatory steroids that also induce biosynthesis of PLA2 inhibitors (Miele L, “New weapons against inflammation: dual inhibitors of phospholipase A2 and transglutaminase”, J. Clin. Invest. 111,19-21,2003). Similarly, protein-based TNF alpha inhibitors, including Etanercept®, Infliximab® and Adalimumab®, have demonstrated efficacy and have been approved for clinical use in various inflammatory diseases. However all these compounds cause systemic potentially serious adverse effects (Palladino M A, Bahjat F R, Theodorakis E A, Moldawer L L, “Anti-TNF-alpha therapies: the next generation”, Nat Rev Drug Discov. 2003;2(9):736-46). An example of the use of interferon antagonists for the treatment of interferon related inflammation disease is described in U.S. Pat. No. 7,285,526. An example of the use of interferon antagonists for the treatment of specific skin conditions characterized by increased T cell activation, e.g. UV damage is disclosed in U.S. Pat. No. 7,323,171. An example of the use of natural TNF alpha inhibitor as a preventive, ameliorating, or therapeutic agent for diseases caused by abnormal production of TNF alpha is disclosed in U.S. Pat. No. 7,199,152.

The avocado fruit is widely consumed as food through the world, and is also used for various medicinal purposes. The health benefits of avocado may be due to the fact that it contains over 20 essential nutrients and various potentially biologically active compounds.

Acetyl derivatives of fatty polyhydroxylated alcohols (PFA) are present both in the avocado pear and in the seeds. Non-acetylated fatty polyhydroxylated alcohol was also detected in avocado in a minor quantity. Acetyl derivatives of fatty polyhydroxylated alcohols are a group of lipids having relatively similar structures. These substances have been previously found to be active against cancer cell lines (Oberlies N H, Rogers L L, Martin J M, McLaughlin J L. “Cytotoxic and insecticidal constituents of the unripe fruit of Persea Americana”. J Nat Prod 1998;61:781-5), and have demonstrated liver protective and anti toxic activity (Kawagishi H, Fukumoto Y, Hatakeyama M, He P, Arimoto H, Matsuzawa T, et al. “Liver injury suppressing compounds isolated from avocado”. J Agric Food Chem 2001;49:2215-21) and moderate activity against epimastigotes and trypomastigotes (Abe F, Nagafuji S, Okawa M, Kinjo J, Akahane H, Ogura T, et al. “Trypanocidal constituents in plants 5. Evaluation of some Mexican plants for their trypanocidal activity and active constituents in the seeds of Persea Americana”. Biol Pharm Bull 2005; 28: 1314-7). In addition, some of these compounds have also demonstrated antifungal (Domergue F, Helms G L, Prusky D, Browse J. “Antifungal compounds from idioblast cells isolated from avocado fruits”. Phytochemistry 2000; 54:183-9) and antibacterial properties (Neeman I, Lishitz, A and Kashman, Y. “New antibacterial agent isolated from avocado pear”. Applied Microbiology. 1970; 19: 470-3), and significant ability to inhibit acety CoA carboxylase activity (Hashimura H, Ueda C, Kawabata J, Kasai T. “Acetyl-CoA carboxylase inhibitors from avocado (Persea americana Mill.) fruits”. Biosci Biotechnol Biochem 2001; 65: 1656-8).

The unsaponifiable fraction of avocado oil is the fraction containing fatty substances, which remain insoluble in water after prolonged hydrolysis in alkaline solution, and could be extracted using organic solvents. The unsaponifiable fraction of avocado and avocado seed oil is being used for several cosmetic and therapeutic applications. For example, PCT Application WO 99/43298 describes the use of a dermatological formulation containing unsaponifiable lipid extract from avocado seed for ameliorating stretch marks and keratosis, such as those due to photo-damage.

During the process of triglyceride isolation by saponification of avocado oil in mineral alkaline solution, acetyl derivatives of Polyhydroxylated Fatty Alcohols (PFA) were found to be hydrolyzed to non-acetylated polyhydroxylated fatty alcohol and to remain in that state in the unsaponifiable fraction.

Another main group of compounds present in avocado seed unsaponifiables are furans (FIG. 4), which may be present in a concentration of up to 30% of the unsaponifiable fraction. The furans compounds have demonstrated biological active properties. U.S. Pat. No. 6,582,688 describes a method for isolation of avocado fractionation of unsaponifiable substances which allows separation of the fraction consisting of furan lipids in a mixture with non-acetylated fatty polyhydroxylated alcohols (up to 25%) and the use of those furan based compounds in cosmetic treatment of the skin and for treatment of inflammatory disorders.

The presence in these applications of both fatty polyhydroxylated alcohols in deacetylated form, and the furan lipids are the major disadvantage of this formulations. Furan lipids are known potent inhibitors of lysyl oxidase an enzyme which is important for normal skin tone and elasticity (M. J. Werman, S. Mokady, and I. Neeman: “Partial Isolation and Characterization of a New Natural Inhibitor of Lysyl Oxidase from Avocado Seed Oil”. J, Agric. Food Chem. 1990;38;2164-2168; Rosenblat G, Kagan H, Shah M, Spiteller G, Neeman I, “Chemical Characterization of Lysyl Oxidase Inhibitor from Avocado Seed Oil”, JAOCS 1995;72:225-9).

Due to the inhibitory effect of the furans on lysyl oxidase activity, furan-containing lipids might potentially serve as anti-fibrotic drugs in the treatment of diseases involving excess collagen and elastin deposition, in scleroderma-related conditions for the inhibition of intra and intermolecular cross-linking, and possibly from enhancing the cleavage of newly-formed cross-links. In contrast, in other conditions, reduced lysyl oxidase activity is associated with increased risk of skin laxity and joint hyper-extensibility (Song Y L, Ford J W, Gordon D, Shanley C J “Regulation of lysyl oxidase by interferon-gamma in rat aortic smooth muscle cells”. Arterioscler Thromb Vasc Biol. 2000; 20:982-88). Using experimental animal models, it was demonstrated that in vivo reduced lysyl oxidase activity is associated with development of aortic aneurysms (Maki J M, Räsänen J, Tikkanen H, Sormunen R, Mäkikallio K, Kivirikko K I, Soininen R. “Inactivation of the lysyl oxidase gene Lox leads to aortic aneurysms, cardiovascular dysfunction, and perinatal death in mice, Circulation”. 2002; 106(19):2503-9).

### SUMMARY OF THE INVENTION

There is thus a need for, and it would be useful to have, a therapeutic composition comprising Polyhydroxylated Fatty Alcohols (PFA), optionally from avocado, for prevention or treatment of skin and inflammatory diseases, for example those diseases caused by increased T cell proliferation and by abnormal production of TNF-alpha, IFN g and PLA 2 activation.

The present invention overcomes these drawbacks of the background art, in at least some embodiments, by providing a composition comprising a pharmaceutically effective amount of at least one isolated polyhydroxylated fatty alcohol or a derivative thereof, which may optionally be an isolated natural PFA or alternatively which may be synthesized, which has a therapeutic effect.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1A and 1B are elution profile by gas chromatography of the natural derivative of polyhydroxylated fatty alcohols from avocado seed (A) and pear (B);

FIG. 2 the structures of major natural derivatives of polyhydroxylated fatty alcohols from avocado;

FIG. 3 illustrates the chemical structure of two de-acetylated derivatives of natural polyhydroxylated fatty alcohols from avocado seed;

FIG. 4 illustrates the chemical structure of representative furan lipid from avocado seed;

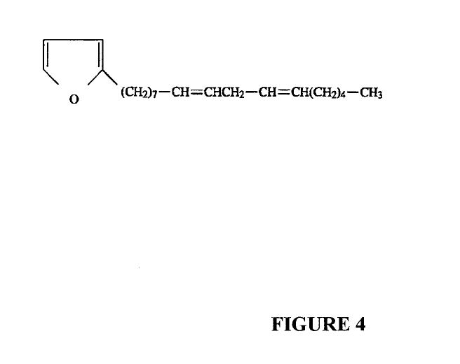

FIG. 5 is a bar chart demonstrating the inhibitory effect of natural polyhydroxylated fatty alcohols on the proliferation of human T cells and Jurkat cells;

FIG. 6 is a bar chart demonstrating the effect of natural derivative of polyhydroxylated fatty alcohols and de-acetylated polyhydroxylated fatty alcohols and on viability of primary T-cells;

FIG. 7 is a bar chart demonstrating the inhibitory effect of natural derivatives of fatty polyhydroxylated alcohols from avocado on TNF-α and IFN-γ secretion by human CD3⁺T lymphocytes after activation by anti-CD3 antibody;

FIG. 8 is a bar chart showing the effect of fatty polyhydroxylated alcohols on 12-O-Tetradecanoylphorbol-13-acetate (TPA) induced IL-6 secretion by human primary keratinocytes;

FIG. 9 is a bar chart showing the effect of fatty polyhydroxylated alcohols on PMA-induced PGE2 secretion by human primary keratinocytes;

FIG. 10 is a bar chart demonstrating the inhibitory effect of polyhydroxylated fatty alcohols and its mixture with ursolic acid and acetyl salicylic acid on prostaglandins (PGE₂) secretion by UV-irradiated primary human keratinocytes;

FIG. 11 Shows an inhibitory effect of PFA on total phospholipase A2 (PLA2) in human primary keratinocytes;

FIG. 12 shows an inhibitory effect of PFA on secretory phospholipase A2 (sPLA2) activity in human primary keratinocytes;

FIG. 13 shows a GC elution profile of acetyenic polyhydroxylated fatty alcohol isolated from mixture of natural PFA;

FIG. 14 shows that acethylenic polyhydroxylated fatty alcohols reduce TPA-induced mouse ear edema development;

FIG. 15 shows that hydrogenated (chemically reduced) polyhydroxylated fatty alcohols affect TPA-induced mouse ear edema differently than acetylenic PFA; and

FIG. 16 shows that acetylenic polyhydroxylated fatty alcohols reduce polymorphonuclear leukocytes influx (myeloperoxidase activity) in mouse-ears treated with TPA.

### DESCRIPTION OF THE PREFERRED EMBODIMENTS

The present invention, in at least some embodiments, is of therapeutic compositions comprising a pharmaceutically effective amount of at least one polyhydroxylated fatty alcohol or a derivative thereof.

According to some embodiments, the PFA comprises one or more isolated natural PFAs. According to other embodiments, the PFA comprises one or more synthetic PFAs. Optionally, combinations of synthetic and natural isolated PFAs may be used.

Polyhydroxylated fatty alcohols (PFA) have significant biological effects on human skin cells and inflammatory cells, and are important for therapeutic treatment or prevention of various skin and/or inflammatory diseases as described herein. By “treatment”, it is meant also prevention and/or amelioration. Various embodiments of the present invention comprise or use these compounds. By “polyhydroxylated fatty alcohols”, it is meant any polyhydroxylated fatty alcohol which may be found in or derived from substances found in any type of fruit or vegetable, preferably avocado seed or the flesh of the avocado fruit. By “derived from”, it is meant any type of derivation of any polyhydroxylated fatty alcohol which may be found in any type of fruit or vegetable, preferably avocado seed or the flesh of the avocado fruit, as described herein.

The present inventors have surprisingly discovered that natural polyhydroxylated fatty alcohols, for example those isolated from avocado and avocado seed, are able to simultaneously inhibit T-lymphocyte proliferation, TNF-alpha and IFN-gamma expression, and PLA2 activity.

According to some embodiments the present invention provides therapeutic topical compositions comprising pharmaceutically effective amounts of polyhydroxylated fatty alcohols or derivatives thereof, and uses thereof.

According to preferred embodiments, polyhydroxylated fatty alcohols or derivatives thereof preferably comprise a backbone of from C13 to C25 carbons, optionally with at least one unsaturated carbon bond. Preferably, if at least one unsaturated carbon bond is present, it is present between the last two carbons of the backbone, whether as a double bond or triple bond. Optionally and more preferably, the hydroxyl groups are present at C1, C2 or C4.

According to other preferred embodiments, derivatives of polyhydroxylated fatty alcohols preferably comprise polyhydroxylated fatty alcohols that have been acylated (esterified) or oxidized or have undergone reaction of the unsaturated carbon bonds with one or more other molecules, for example for hydrogenation of the unsaturated carbon bonds.

According to other preferred embodiments, derivatives of polyhydroxylated fatty alcohols preferably comprise polyhydroxylated fatty alcohols that have been acylated (esterified) or oxidized or have undergone reaction of the unsaturated carbon bonds with one or more other molecules, for example for hydrogenation of the unsaturated carbon bonds.

The term “natural derivatives of fatty polyhydroxylated alcohols” as used herein refers to all types of derivatives of fatty polyhydroxylated alcohols which are present in fruit or vegetable extracts and which have not undergone hydrolysis.

The term “natural derivatives of fatty polyhydroxylated alcohols” as used herein refers to all types of derivatives of fatty polyhydroxylated alcohols which are present in fruit or vegetable extracts and which have not undergone hydrolysis.

As used herein, “natural” includes all materials from the extract, including acetylated, with a minor component of non-acetylated. After the hydrolysis process, the fatty alcohols become de-acetylated and are not referred to as natural herein.

The term “acetylated polyhydroxylated fatty alcohols” as used herein refers to all types of derivatives of polyhydroxylated fatty alcohols containing at least one acetyl group instead of a hydrogen atom in a hydroxyl group. The acetyl group may optionally be at C1, C2 or C4, but is preferably at C1 or C4.

Furthermore, it has surprisingly been demonstrated that acetylated derivatives of polyhydroxylated fatty alcohols have significantly less toxicity compared to non-acetylated polyhydroxylated fatty alcohols, which are formed in the process of saponification.

Alkaline hydrolysis of polyhydroxylated fatty alcohols leads to formation of mainly 1,2,4-Trihydroxyheptadeca-16-ene and 1,2,4-Trihydroxyheptadeca-16-yne, the cytotoxicity of the hydrolyzed compounds mixture is significantly higher than that of their natural acetylated derivatives.

Hence there is a significant advantage to using naturally occurring acetylated polyhydroxylated fatty alcohols instead of de-acetylated compounds separated from unsaponifiables, or in combination with other avocado unsaponifiables as described in PCT Application WO 99/43298 and U.S. Pat. No. 6,582,688. Moreover, the presence at least one acetyl group in the structure of derivatives of polyhydroxylated fatty alcohols increases the stability of the product and protects the active molecule against oxidation.

As discussed in the Background section above, the presence of the furan lipids in compositions comprising polyhydroxylated fatty alcohols is disadvantageous due to the inhibitory effect of the furan lipids on lysyl oxidase activity.

Thus, it would clearly be highly beneficial to provide compositions comprising polyhydroxylated fatty alcohols, which are free or substantially free of such furan compounds to avoid possible negative effect of the compounds for therapeutic applications. Similarly, the use of the natural polyhydroxylated fatty alcohol is highly preferable to use of deacetylated derivatives to avoid the effects, which are associated with the increased cytotoxicity of the deacetylated compounds.

According to some embodiments, the derivative of natural polyhydroxylated fatty alcohols substantially comprises an acetylated derivative.

The term “acetylated fatty polyhydroxylated alcohols” as used herein refers to all types of derivatives of fatty polyhydroxylated alcohols containing at least one acetyl group instead of a hydrogen atom in a hydroxyl group.

According to still further features in the described preferred embodiments, the composition is free or completely free of furan lipids. By “completely free of furan lipids”, it is meant that up to about 5% of furan lipids may be present in the composition. By “free of furan lipids” it is meant that up to about 20%, preferably up to about 15%, more preferably up to about 10% and most preferably up to about 7.5% of furan lipids may be present in the composition.

According to some embodiments, the fatty polyhydroxylated alcohols are isolated or synthesized in substantially pure form, such as, for example, 95% pure, 90% pure, 85% pure or 80% pure. Optionally and preferably, the polyhydroxylated fatty alcohol is present in a concentration of from about 80% to about 95% w/w of the isolated material.

According to some embodiments of the present invention, the isolated polyhydroxylated fatty alcohols are isolated from a fruit or vegetable source. Optionally and preferably, the fruit or vegetable comprises avocado fruit and/or avocado seed.

According to some embodiments of the present invention, the polyhydroxylated fatty alcohols or derivatives thereof include but are not limited to, 1,2,4-Trihydroxyheptadecan, 1,2,4-Trihydroxyheptadeca-16-ene, 1,2,4-Trihydroxyheptadeca-16-yne, 1-Acetoxy-2,4-dihydroxyheptadeca-16-ene, 1-Acetoxy-2,4-dihydroxyheptadeca-16-yne, 4-Acetoxy-1,2-dihydroxyheptadeca-16-ene, 4-Acetoxy-1,2-dihydroxyheptadeca-16-yne or combinations thereof.

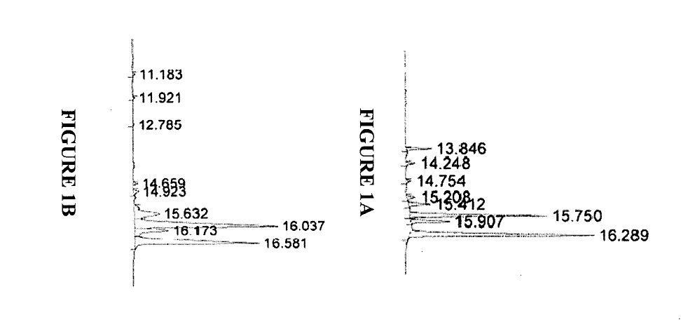

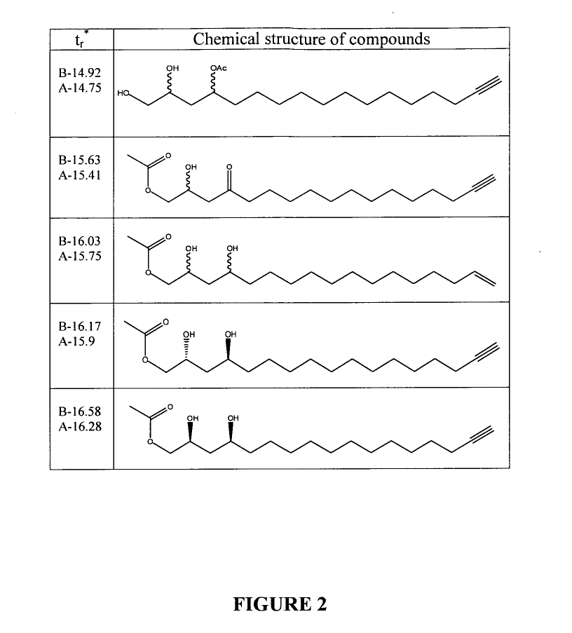

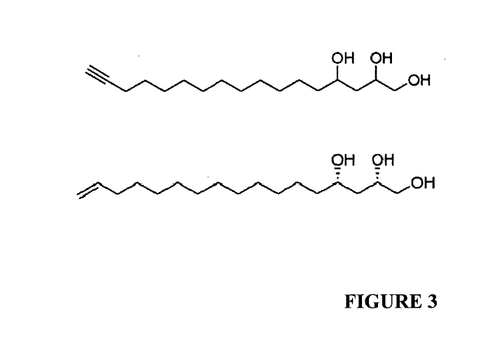

Examples of structures of some natural lipids isolated from avocado are shown in FIG. 1. The elution profile and structures of the main and some minor derivatives of natural acetylated fatty polyhydroxylated alcohols that were separated from avocado seed are shown in FIG. 1, while FIG. 2 shows representative structures of polyhydroxylated fatty alcohols obtained by saponification of acetylated polyhydroxylated fatty alcohols in alkaline solution. FIG. 3 demonstrated the structure of representative furan lipids from avocado seed, thereby showing the differences in structure from the preferred embodiments of polyhydroxylated fatty alcohols of the present invention.

According to at least some embodiments of the present invention, the composition is adapted for topical administration. Optionally, the composition further comprises a pharmaceutically acceptable carrier. Examples of suitable carriers include water; vegetable oils; mineral oils; esters such as octal palmitate, so isopropyl myristate and isopropyl palmitate; ethers such as dicapryl ether and dimethyl isosorbide; alcohols such as ethanol and isopropanol; fatty alcohols such as cetyl alcohol, cetearyl alcohol, stearyl alcohol and biphenyl alcohol; isoparaffins such as isooctane, isododecane and is hexadecane; silicone oils such as cyclomethicone, dimethicone, dimethicone cross-polymer, polysiloxanes and their derivatives, preferably organomodified derivatives; hydrocarbon oils such as mineral oil, petrolatum, isoeicosane and polyisobutene; polyols such as propylene glycol, glycerin, butylene glycol, pentylene glycol and hexylene glycol; waxes such as beeswax and botanical waxes; or any combinations or mixtures thereof.

The composition may also have one or more of the following optional additional ingredients: anesthetics; anti-allergenics; antimicrobials; antifungals; anti-inflammatories; antiseptics; depigmenting agents; sunscreens; antioxidants.

Examples of antioxidants capable of slowing or preventing the oxidation process include compounds such as green tea based polyphenols, Coenzyme Q10 (CoQ10), glutathione, vitamin C, Vitamin A, Lycopene, Carotenoids, Flavonoids/polyphenols and vitamin E as well as enzymes such as catalase, and peroxidase.

Other particularly useful additional ingredients are anti-inflammatories. The anti-inflammatories may be of synthetic, natural, or semi-synthetic origin. The anti-inflammatories may be steroidal or non-steroidal. Useful examples include, but are not limited to, mangostin, eysenhardtia polistachya (Palo Azul) wood extract, rosemary extract, camphor, salicylates, hydrocortisone, aspirin, indomethacin, mefenamic acid and derivatives thereof.

Other particularly useful additional ingredients are sunscreens. Preferred sunscreens are those with a broad range of UVB and UVA protection, such as octocrylene, avobenzone (Parsol 1 78 9), octyl methoxycinnamate, homosylate, benzophenone, camphor derivatives, zinc oxide, and titanium dioxide.

Other particularly useful additional ingredients are exfoliating agents, such as alphahydroxyacids, betahydroxyacids, oxa acids, oxa diacids, and their derivatives such as esters, anhydrides and salts thereof.

The composition may further optionally comprise one or more pharmaceutically acceptable excipients, including but not limited to water soluble colorants (such as FD&C Blue #1); oil soluble colorants (such as D&C Green #6); chelating agents (such as Disodium EDTA); emulsion stabilizers (such as carbomer); preservatives (such as Methyl Paraben); fragrances (such as pinene); flavoring agents (such as sorbitol); humectants (such as polyethylene glycol, propylene glycol, glycerin, 1,3-butylene glycol, hexylene glycol, xylitol, sorbitol, maltitol, chondroitin sulfuric acid, hyaluronic acid, mucoitin sulfuric acid, caronic acid, Atelocollagen, cholesteryl 12-hydroxystearate, sodium lactate, bile salts, dl-pyrrolidonecarboxylic acid salts, short chain soluble collagen, diglycerol (ethylene oxide) propylene oxide adduct, extract of chestnut rose, extract of malifoils (Achillea millefolium), and extract of melilots); whitening agents (such as placental extract, glutathione, extract of creeping saxifrage (Saxifrage stolonifera), waterproofing agents (such as PVP/Eicosene Copolymer); water soluble film-formers (such as Hydroxypropyl methylcellulose); oil-soluble film formers (such as Hydrogenated C-9 Resin); cationic polymers (such as Polyquatemium 10); anionic polymers (such as xanthan gum); emollients, such as dimethicone, polysilicones and cyclomethicone; lubricants; moisturizers; skin penetration enhancers; surfactants; thickeners; viscosity modifiers; and the like.

The effective amount of isolated natural polyhydroxylated fatty alcohols or derivatives thereof and the duration of application of the composition will vary with the particular condition being reduced or prevented, the particular carrier utilized, and like factors in the knowledge and expertise of those skilled in the art.

According to some embodiments of the present invention, the natural polyhydroxylated fatty alcohol is present in a concentration of from about 0.001% to about 20% w/w of the total composition.

The duration of application may be, for example, once or twice a day for a period of at least one week, two weeks or more.

The composition of the present invention can be made into any suitable product form, such as aerosol, cake, cream, ointment, emulsion, essence, foam, gel, lotion, mousse, paste, patch, pencil, serum, solution, towelette, mask, body wrap, spray and stick.

According to other embodiments of the present invention, the pharmaceutical composition is adapted for systemic administration.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the preparation accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases. One of the ingredients included in the pharmaceutically acceptable carrier can be for example polyethylene glycol (PEG), a biocompatible polymer with a wide range of solubility in both organic and aqueous media.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intravenous, intraperitoneal, intranasal, or intraocular injections. Alternately, one may administer a preparation in a local rather than systemic manner, for example, via injection of the preparation directly into a specific region of a patient's body.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the invention may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The preparations described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The preparation of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of active ingredients effective to prevent, alleviate or ameliorate symptoms of disease or prolong the survival of the subject being treated.

Determination of a therapeutically effective amount is well within the capability of those skilled in the art.

For any preparation used in the methods of the invention, the therapeutically effective amount or dose can be estimated initially from in vitro assays. For example, a dose can be formulated in animal models and such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human.

The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p.1).

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions including the preparation of the present invention formulated in a compatible pharmaceutical carrier may also be prepared, placed in an appropriate container, and labeled for treatment of an indicated condition.

Pharmaceutical compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert.

Ultraviolet radiation is harmful to a wide range of biological systems. The extent of damage depends upon the level and duration of exposure as well as the susceptibility and resilience of the exposed organism. The key human health effects from exposure to UV radiation include skin cancer, cataracts, and immunosuppression. In addition, other dermatological effects include severe photo-allergies and accelerated aging of the skin. Damage to the skin by UV radiation reduces its immunological defenses, impeding resistance to infectious diseases as well as to skin tumors, and diminishing the effectiveness of vaccines.

Due to their ability to inhibit T-lymphocyte proliferation, TNF-alpha and IFN-gamma expression and PLA2 activity, the compositions of the present invention, in at least some embodiments are able to treat or prevent a large number of immune disorders and inflammatory conditions.

According to some embodiments, the compositions of the present invention are useful for preventing and/or ameliorating pre-cancerous damage to the skin caused by ultraviolet radiation.

According to some embodiments, the compositions of the present invention are useful for treating and/or preventing the degenerative effects of UV radiation in skin.

According to some embodiments, the compositions of the present invention are useful for treating and/or preventing contact dermatitis.

According to some embodiments, the compositions of the present invention are useful for treating and/or preventing atopic dermatitis.

According to some embodiments, the compositions of the present invention are useful for treating and/or preventing psoriasis.

According to some embodiments, the compositions of the present invention are useful for treating and/or preventing skin inflammatory disorders.

According to some embodiments, the compositions of the present invention are useful for the prevention of cancer, such as skin cancer, including but not limited non-melanoma skin cancer.

According to some embodiments, non melanoma skin cancer treatable and/or preventable by use of the compositions of the present invention includes basal cell carcinoma and squamous cell carcinoma.

According to some embodiments, the compositions of the present invention are useful for treating and/or preventing skin cancer caused, for example, by ultraviolet irradiation, immunosuppression, x-irradiation, or by exposure to a chemical (such as arsenic or a hydrocarbon).

According to some embodiments, the compositions of the present invention are useful for treating and/or preventing atherosclerosis.

According to some embodiments, the compositions of the present invention are useful for treating and/or preventing inflammatory bowel disease. According to some embodiments, the compositions of the present invention are useful for treating and/or preventing arthritis.

According to some embodiments, the compositions of the present invention are useful for treating and/or preventing neurodegenerative disorders.

According to some embodiments, the compositions of the present invention are useful for treating and/or preventing paradontosis.

According to some embodiments, the compositions of the present invention are useful for treating and/or preventing asthma.

According to some embodiments, the compositions of the present invention are useful for treating and/or preventing autoimmune diseases, such as for example Crohn's disease.

The present invention further provides a method for the isolation of at least one natural polyhydroxylated fatty alcohol or a derivative thereof, preferably for the preparation of a topical composition. The method preferably comprises specific isolation of the fraction of natural derivatives of fatty polyhydroxylated alcohols from a fruit or vegetable source, such as from crude extract of avocado seed.

The process optionally and more preferably includes the stage of crushing and lyophilizing the fruit or vegetable source. The lyophilized powder is optionally and preferably extracted using a non-polar (organic) solvent (e.g. hexane, petroleum ether) or polar (ethanol, methanol), to obtain a crude lipid extract. The crude lipid extract is concentrated by using a non-polar solvent (e.g. hexane, petroleum ether) or a polar solvent (e.g. ethanol, methanol). The desired components are preferably separated from the concentrated crude lipid extract by cool crystallization, i.e. crystallization at a temperature which is lower than room temperature, followed by filtration. Filtered compounds are dissolved in ethanol, and insoluble, highly non-polar compounds are separated by filtration. Ethanol is evaporated and the compounds obtained are re-crystallized with a non-polar solvent, such as, for example, hexane or petroleum ether.

Optionally and preferably, the fruit or vegetable source comprises avocado fruit and/or avocado seed.

Surprisingly, it was found that the compounds that are re-crystallized from cooled avocado seed extract in a non-polar solvent are enriched with acetylated polyhydroxylated fatty alcohols and do not include furan-containing lipids, or contain those compounds only a minor trace amount.

This method of isolation of natural derivatives of polyhydroxylated fatty alcohols significantly increases the concentration of these active compounds, by at least about four times, compared to background art methods using molecular distillation, such as described in patent U.S. Pat. No. 6,582,688, which results in a concentration of up to 25% polyhydroxylated fatty alcohols in a mixture with furan containing lipids.

Natural derivatives of polyhydroxylated fatty alcohols separated by the method of the present invention may comprise up to 95% by weight dry powder.

Natural derivatives of polyhydroxylated fatty alcohols separated by this method may comprise up to 95% by weight dry powder. The composition of the present invention may optionally comprise from about 0.01% to about 90% by weight of natural polyhydroxylated fatty alcohols.

The method of isolation of natural fatty polyhydroxylated alcohols and derivatives described above may be used for the isolation of inhibitors of an inflammatory process such as T lymphocyte proliferation, TNF alpha and IFN-γ expression and phospholipase A, from a fruit or vegetable source. Optionally and preferably the fruit or vegetable source comprises avocado fruit and/or avocado seeds.

The ability of polyhydroxylated fatty alcohols to inhibit PLA2 activity and T lymphocyte proliferation and TNF alpha and IFN gamma expression has been demonstrated in vitro as shown in below

### EXAMPLES

Reference is now made to the following examples, which together with the above description; illustrate the invention in a non limiting fashion.

### Example 1

### Isolation of-natural Polyhydroxylated Fatty Alcohols from Avocado Seeds and their Alkaline Hydrolysis

Avocado seeds were separated from the avocado pear followed by freezing and lyophylization. 10 kg of lyophilized and powdered seed was consequently extracted using hexane in Soxhlet apparatus for 14 h.

Organic solvent was evaporated in a rotor evaporator at temperature intervals of 40-60° C., at a pressure of about 30 millibar. Extracted compounds were re-dissolved with two volumes of hexane or petroleum ether (as a non-limiting example of a non-polar solvent) and then were put into a cold room having a temperature in the range of 2-8° C. for about 12 hours for the process of cool crystallization.

Crystallized compounds were separated from the solvent by filtration in Worthman filter paper.

The process yielded 30 g of crystalloid compounds. GC elution profile and chemical structure according to GC/MS and HPLC/MS-ECI analysis are presented in FIGS. 1A and 2. No furan lipids were detected.

De-acetylated PFA were obtained by alkaline hydrolysis of natural PFA in 2% Sodium hydroxide in methanol at room temperature for 24 h.

At the end of reaction ethanol solution was neutralized by 5% hydrochloric acid and de-acetylated polyhydroxylated fatty alcohols were extracted by diethyl ether. The solvent was removed under reduced pressure.

### Example 2

### Isolation of Fatty Polyhydroxylated Alcohols from Avocado Pear

In order to isolate polyhydroxylated fatty alcohols in the edible part of the avocado fruit, 200 g ground avocado pear (Hagalil or Ettinger) were extracted twice in 400 ml heated ethanol at 60° C. for 1 h, followed by acetone extraction at 4° C. overnight. All extracts were collected and the solvents were evaporated.

Dried extract was re-dissolved in 35 ml hexane. Avocado pear hexane extract was refrigerated (4° C.) overnight and precipitated polyhydroxylated fatty alcohols were separated by filtration.

The process yielded 100-140 mg of crystalloid compounds. Elution profile by GC and chemical structure according to GC/MS analysis are presented in FIGS. 1B and 2.

### Example 3

### Effect of Polyhydroxylated Fatty Alcohols on T-cells and Jurkat Cell Proliferation and on T-cells Viability

Human T cells were purified from peripheral blood of healthy human donors. The whole blood was incubated (20 min, 22° C.) with RosetteSep™ human T-cell enrichment cocktail (StemCell Technologies, Vancouver, BC, Canada). The remaining unsedimented cells were then loaded onto Lymphocyte Separation Medium (ICN Biomedicals; Belgium), isolated by density centrifugation, and washed with PBS. The purified cells (>95% CD3⁺ T cells) obtained were cultured in RPMI containing antibiotics and 10% heat-inactivated FCS.

Proliferation of T-cells was assessed by the 2,3-bis[2-methoxy-4-nitro-5-sulfophenyl]-2H-tetrazolium-5-carboxanilide (XTT) assay after mitogenic anti-CD3 cells activation in presence PFA.

For the study the effect of polyhydroxylated fatty alcohols on T cell viability, CD3 T cells were incubated with PFA for 72 hours and after this incubation, T cell viability was defined by XTT assay.

Activation of CD3⁺ T-cells by addition of anti-CD3 stimulates T-cells proliferation. This proliferation, nevertheless, was inhibited by pre incubation of the cells with PFA. About 40% growth inhibition was observed in anti-CD3 activated T-cells that were treated with natural PFA at concentration 10 μg/ml.

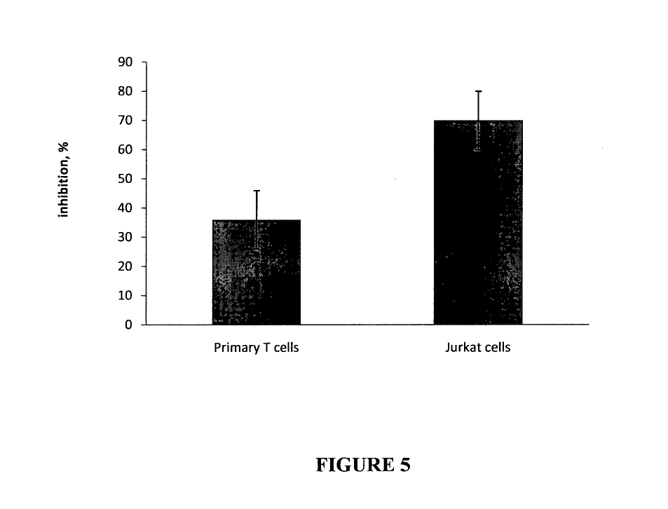

Jurkat cells were more susceptible to PFA, achieving more than 80% inhibition of cell proliferation in similar condition. The results are shown in FIG. 5.

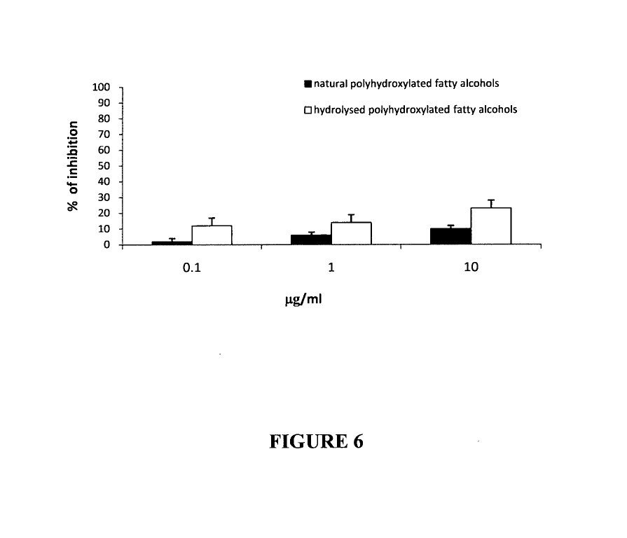

Viability assay revealed slightly decreased number of T-cells after 72 h administration with PFA, compared with non-treated cells At PFA concentration of 10 μg/ml the number of viable cells was decreased by less than 10% (FIG. 6), that did not account for the 40% of T-cell inhibition proliferation (FIG. 5).

De-acetylated PFA at concentration of 10 μg/ml significantly decrease T-cell proliferation (about 20% inhibitions) (FIG. 6).

### Example 4

### Inhibitory Effect of Natural-polyhydroxylated Fatty Alcohols from Avocado Seeds on TNF alpha and INF gamma Production by T Lymphocytes

The following experiment was performed in order to confirm the inhibitory effect of natural derivatives of fatty polyhydroxylated alcohols from avocado seeds on TNF alpha and INF gamma production by T lymphocytes and on the viability of those cells.

For cytokine secretion, T cells (2×10⁶ cells per ml) were activated (1 hr, 37° C.) with the indicated concentrations of reagents in 24-well plates in media based on RPMI containing 10% heat-inactivated FCS. The cells were then washed and re-plated at the same concentration on anti-CD3 mAb pre-coated 24-well plates (2 μg/ml; non tissue culture grade plates), at 4° C. for 24 hr with and without fatty polyhydroxylated alcohols from avocado seeds. The supernatants were collected, and the cytokine content (TNF-α, IFN-γ) was determined by ELISA commercial kits (OptiEIA kits; BD Pharmingen) according to the manufacturer's instructions.

Pre-treatment of the cells with PFA caused significant dose-dependent suppression of TNF alpha and IFN gamma secretion by the activate T-cells.

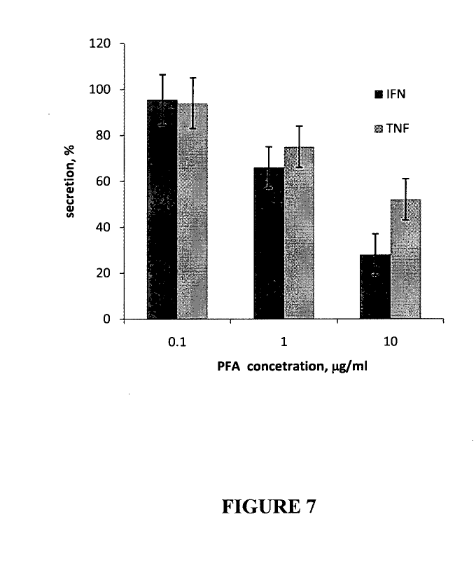

TNF alpha and IFN gamma secretion by T-cell pre-treated with PFA at concentration 1 μg/ml was 25% and 30% below than that in control cells. Suppression of cytokine expression in presence of PFA at concentration of 10 μg/ml was much higher achieving of about 50% and 70% correspondingly. The results are shown in FIG. 7.

### Example 5

### Effect of Polyhydroxylated Fatty Alcohols from Avocado Seeds on TPA-induced IL-6 Secretion in Primary Human Keratinocytes

The effect of PFA on TPA-induced secretion of IL-6 in cells were studied in primary human keratinocytes. Primary normal human epidermal keratinocytes were provided by M. Chaouat M.Sc. (Laboratory of Experimental Surgery, Hadassah Hospita, Jerusalem, Israel). Briefly, a thin split thickness skin biopsy is taken asceptically and trypsinized overnight at 4-8° C. The epidermal layer is separated from the dermal layer and the single cells are isolated and cultured in specialized keratinocyte medium. The keratinocytes are then redistributed into the flasks containing lethally irradiated 3T3. The flasks are incubated at 37° C. 8-10% CO₂. Upon reaching subconfluency, the cells are redistributed to new flasks without the 3T3 feeder layer.

Primary human keratinocytes at sub-confluent conditions in 24-well plate were treated with polyhydroxylated fatty alcohols in growth medium for 60 min. At the end of the time, the growth medium was additionally supplemented with 1 ng/ml TPA and the cells were incubated at 37° C. 8-10% CO₂ for more than 8 hours. IL-6 was quantified in the growth medium by ELISA method by using a commercial kit (Human IL-6 Quantikine HS ELISA Ki, R&D system. MN, U.S.A.), according to the manufacturer's instructions.

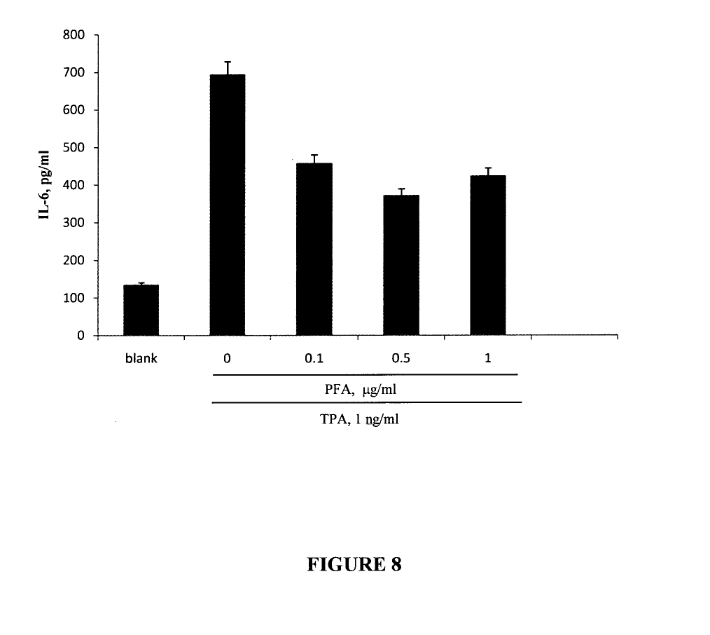

Results are shown in FIG. 8. Pre-treatment of the cells with PFA significantly inhibited secretion of IL-6 by about 40-60%.

### Example 6

### Effect of Polyhydroxylated Fatty Alcohols from Avocado on TPA-induced PGE2 Secretion in Human Primary Keratinocytes

In order to study the effect of PFA on TPA-induced secretion of IL-6, primary human keratinocytes at sub-confluent conditions were treated with PFA in growth medium for 60 min At the end of the time, the growth medium was additionally supplemented with 1 ng/ml TPA and the cells were incubated at 37° C., 5% CO₂ for more than 8 hours PGE2 concentration in the growth medium was quantified. by ELISA method by using a commercial kit (Prostaglandin E2 Parameter Assay Kit, R&D system, MN, U.S.A.), according to the manufacturer's instructions.

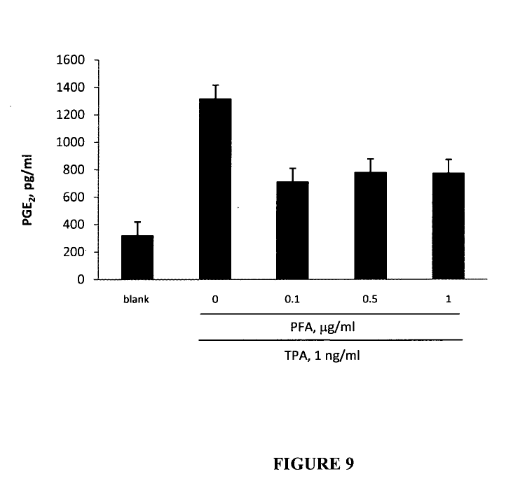

Results are shown in FIG. 9. Pre-treatment of the cells with PFA significantly inhibit TPA-induced secretion of PGE2 by about 40-60%.

### Example 7

### Inhibitory Effect of Polyhydroxylated Fatty Alcohol and its Mixture with Ursolic Acid or Acetyl Salicylic Acid on PGE2 Secretion in UV-irradiated Primary Human Keratinocytes

Primary human keratinocytes at sub-confluent conditions in 24 well plate were treated with ethanol solution of polyhydroxylated fatty alcohol or ursolic acid or acetyl salicylic acid, or with a mixture of polyhydroxylated fatty alcohol and ursolic acid or acetyl salicylic acid in growth medium based on DMEM for 60 min. After removing the media, the cultures were washed thoroughly with PBS, filled with a 1-cm layer of PBS, and irradiated with UVB (30 mJ/cm²). After irradiation of the cells, PBS saline was changed to growth medium containing the test compounds or their mixtures and cells were incubated at 37° C., 5% CO₂ for 8 hours.

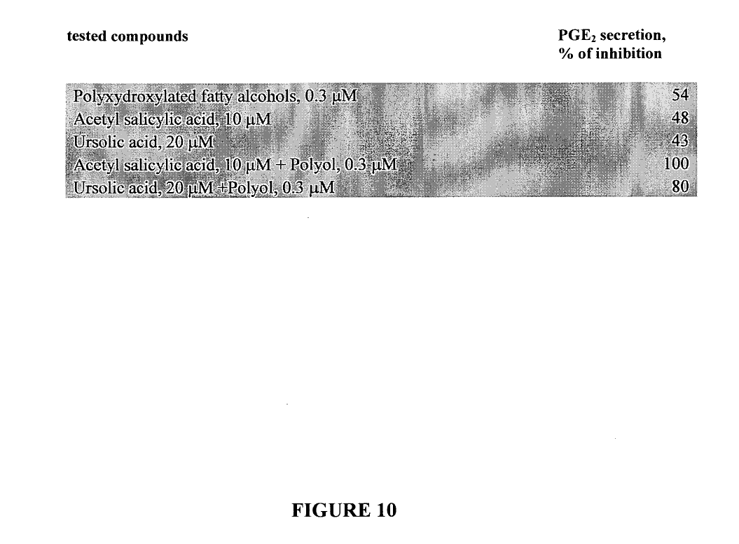

PGE2 was quantified in medium by ELISA method by using the same 1 kit, according to the manufacturer's instructions. The results are shown in FIG. 10.

As FIG. 10 demonstrates, there is a synergetic effect between the biological activities of polyhydroxylated fatty alcohols and COX inhibitors such as acetyl salicylic or ursolic acid. The mixtures of polyhydroxylated fatty alcohols with COX inhibitors were found to decrease PGE2 secretion by primary human keratinocytes at higher level, comparing to the PGE2 secretion inhibitory ability of any ingredient alone.

### Example 8

### Effect of PFA on Total Phospholipase A2 (PLA2) and Secretory Phospholipase A2 (sPLA2) Activity in HaCaT Cells

For testing total PLA2 activity, HaCaT cells were seeded in 24-round multi-well plates. Confluent cells were overnight labeled with [³H]-arachidonic acid (AA) in culture medium based on DMEM. After labeling, the cells were washed from excess free AA with PBS containing fatty acid-free BSA (2 mg/ml) in order to remove the unincorporated radioactivity. The cells were allowed to equilibrate at 37° C. for 1 h with addition of PFA.

After removing the growth media, the cultures were washed thoroughly with PBS, filled with a 1-cm layer of PBS, and were stimulated by irradiation with UVB (60 mJ/cm²). After irradiation of the cells, PBS saline was changed to growth medium containing corresponding concentrations of polyhydroxylated fatty alcohols and the release of H³-AA into the culture medium was monitored for up to two hours. The medium radioactivity is determined by scintillation counter.

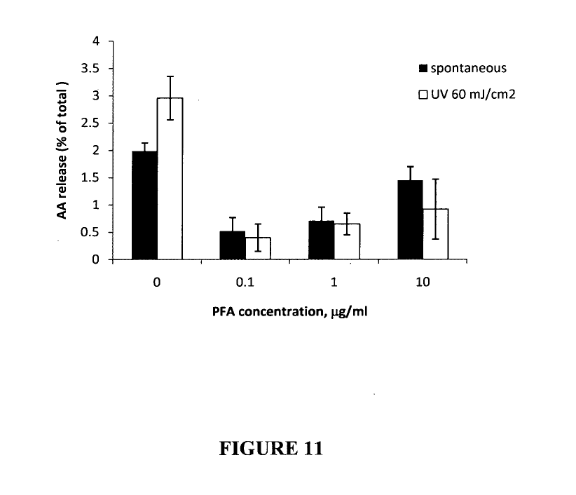

The results are given as released [H³] AA in the supernatant relative to [H³]AA incorporated into the cells (FIG. 11).

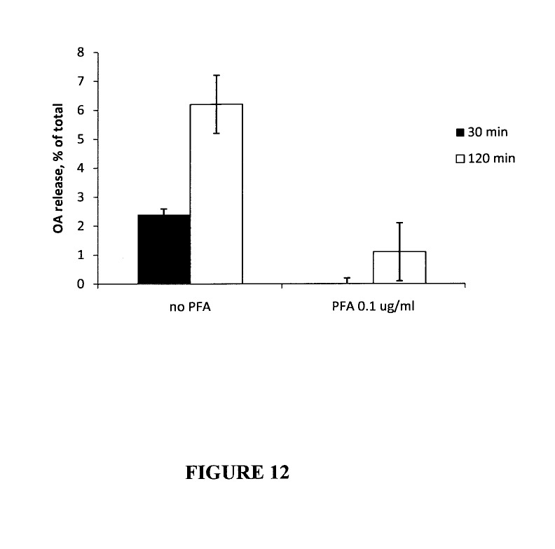

For testing sPLA2 activity, the same metabolic labeling procedure was used with a difference that [3H]-oleic acid was used instead of [3H]-AA. The results are shown in FIG. 12

The results demonstrated in FIGS. 11 and 12, revealed significant inhibition of total PLA2 and sPLA2 activity in the cell treated with PFA at the concentration range of 0.1-1 μg/ml

### Example 9

### Separation of Acetylenic Polyhydroxylated Fatty Alcohols from the Mixture of Natural PFA

500 mg of natural PFA were dissolved in EtOH (abs, 60 ml) and was stirred for 2 hr with AgNO₃, (400 mg) dissolved in water (10 ml). The precipitate obtained was separated by centrifugation and was washed several times with EtOH.

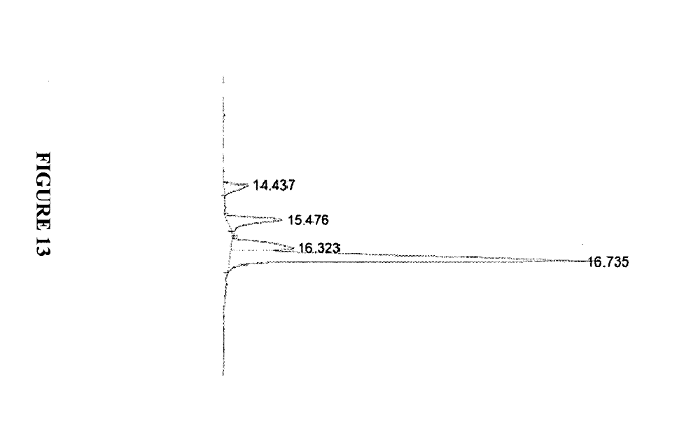

The wet acetylide was suspended and stirred in 5% ammoniumthiocyonate (20 ml) for 48 hr after which it was extracted with ether. The etheral extract was washed with a water solution of NaCl dried under Na₂SO₃ and evaporated, yielding the acetylenic component (200 mg). Elution GC profile of acetylenic PFA is shown in FIG. 13.

### Example 10

### Effect of Acetylenic and Chemically Reduced Polyhydroxylated Fatty Alcohols on TPA-induced Dermatitis (Edema) in Mouse Ear Skin

Edema was induced in the right ear by topical application of 2,5 μg/ear of 12-O-Tetradecanoylphorbol 13-acetate (TPA) dissolved in 20 μL of acetone. The PFA at different amount, as well as dexamethasone and indomethacin (used as a positive control) were applied topically simultaneously with the TPA. Ear thickness was measured before and 6 h after the induction of inflammation.

Edema was expressed as an increase in ear thickness due to the inflammatory challenge. Ear thickness was measured before and after induction of the inflammatory response using a digital micrometer (Great, MT-045B). The micrometer was applied near the tip of the ear just distal to the cartilaginous ridges and the thickness was recorded in μm. To minimize technique variations, a single investigator performed the measurements throughout each experiment. Extracts were applied topically in 20 μL, acetone

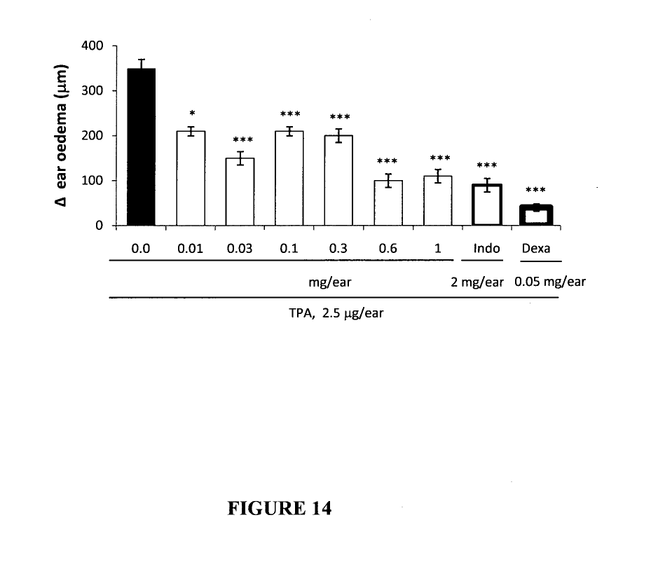

As demonstrated in FIG. 14, acetylenic PFA exhibited strong anti-inflammatory effect achieving ID₅₀ at concentration 30 μg/ear, and maximal inhibition of 72% at 600 μg/ear that is comparable with activity of potent anti-inflammatory drug indomethacin.

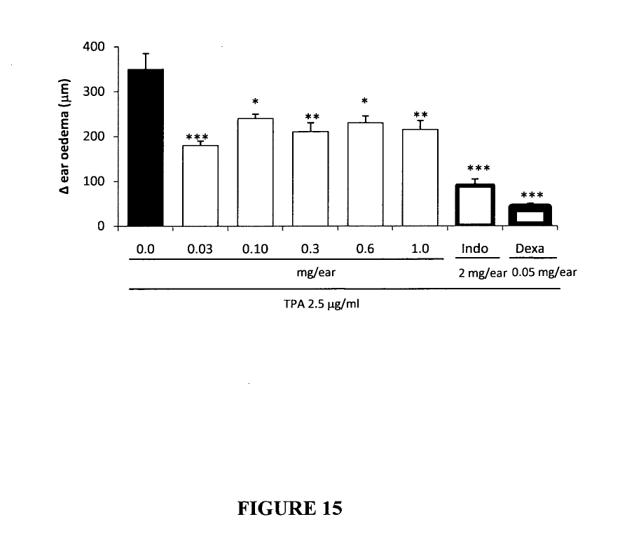

Chemically reduced PFA still demonstrated anti-inflammatory activity at low concentration (FIG. 15). But compared to acetylenic PFA, anti-inflammatory activity of chemically reduced PFA was considerably less strong at higher concentration up to 1 mg/ear.

### Example 11

### Tissue Myeloperoxidase Activity Assay in TPA-induced Dermatitis (Edema) in Mouse Ear Skin

The activity of tissue myeloperoxidase (indicative of polymorphonuclear leukocytes influx) was assessed 24 h after TPA application to the mouse ear.

A biopsy (6 mm ear tissue punch) was placed into 0.75 mL of 80 mM phosphate-buffered saline (PBS), pH 5.4, containing 0.5% hexadecyltrimethylammonium bromide, then homogenized (45 s at 0° C.) with a motor-driven homogenizer. The homogenate was decanted into a microfuge tube, and the vessel was washed with a second 0.75 mL volume of hexadecyltrimethylammonium bromide in buffer. The wash was added to the tube and the 1.5 mL sample was centrifuged at 12,000×g at 4° C. for 15 min. Triplicate 30 μL samples of the resulting supernatant were added to 96-well μL plates.

For the assay, 200 μL of a mixture containing 100 μL of 80 mMPBS, pH 5.4, 85 μL of 0.22M PBS, pH 5.4, and 15 μL of 0.017% hydrogen peroxide were added to the wells. The reaction was started by the addition of 20 μL of 18.4 mM tetramethylbenzidine HCl in dimethylformamide. Plates were incubated at 37° C. for 3 min and then placed on ice, the reaction was stopped by the addition of 30 μL of 1.46M sodium acetate, pH 3.0. Enzyme activity was determined colorimetrically using a plate reader (EL808-BioTech Instruments, Inc.) set to measure absorbance at 620 nm and expressed as mOD/mg tissue.

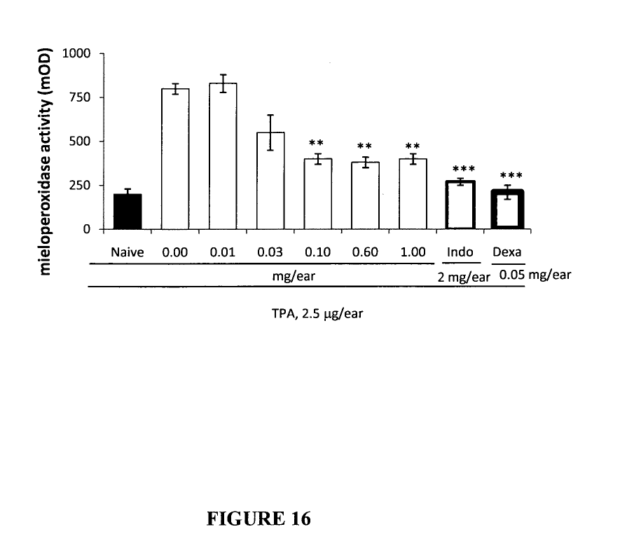

As demonstrated in FIG. 16 acetylated PFA decreased tissues myeloperoxidase activity (indicative of polymorphonuclear leucocytes influx) in mouse-ears treated with TPA with a maximum inhibition of about 57% at 100 μg/ear.

### Example 12

### Chemical Reduction (Hydrogenation) of Unsaturated Polyhydroxylated Fatty Alcohols

The below exemplary, illustrative method relates to the hydrogenation of polyhydroxylated fatty alcohols. A suspension of 0.9 g polyhydroxylated fatty alcohols and 0.28 g Pd/C catalyst in 50 ml absolute ethanol was placed in to 250 ml autoclave and have been mixing under hydrogen (H2) pressure (5 bar) for 24 h. During the reaction of hydrogen and PFAs at room temperature, the pressure of the hydrogen in autoclave was reduced from 5 to 3.7. About 40% of hydrogen was absorbed by unsaturated molecules (presumably by acetylenic PFA) for first 15 min. After passing argon through the reaction mixture, the solution was filtered through the celite filter and ethanol was evaporated under low pressure.

The dried compound was re-dissolved in hexane containing 20% of ethyl acetate and was passed through a SiO2 column (Silica gel 60, 0.063-0.2 mm).The compounds of interest were re-crystallized in heptane.

The yield of the final compounds was 0.6 g (about 66% of initial amount). Chemical reduction of unsaturated polyhydroxylated fatty alcohols from avocado caused the formation of 1-Acetoxy-2,4-dihydroxy-heptadecan.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

Citation or identification of any reference in this application shall not be construed as an admission that such reference is available as prior art to the present invention.

To the extent that section headings are used, they should not be construed as necessarily limiting.

1. (canceled)

2. A method for treating a subject for an inflammatory disease in need thereof, the method comprising administering a pharmaceutical composition comprising polyhydroxylated fatty alcohols or derivatives thereof in a pharmaceutically effective amount, in a pharmaceutically acceptable carrier; wherein said polyhydroxylated fatty alcohols or derivatives thereof comprise a backbone of from C13 to C25 carbons, hydroxyl groups present at one or more of C1, C2 or C4, and at least one unsaturated carbon bond; wherein said at least one unsaturated carbon bond is present between the last two carbons of the backbone; wherein said composition is substantially free of furan lipids.

3. (canceled)

4. (canceled)

5. (canceled)

6. (canceled)

7. The method of claim 2, wherein said polyhydroxylated fatty alcohols are isolated natural polyhydroxylated fatty alcohols.

8. The method of claim 7, wherein said isolated natural polyhydroxylated fatty alcohols are isolated from a fruit or vegetable source.

9. The method of claim 8, wherein said fruit or vegetable source is selected from the group consisting of avocado fruit and avocado seed.

10. The method of claim 2, wherein said derivative of natural polyhydroxylated fatty alcohols substantially comprises an acetylated derivative.

11. The method of claim 2. wherein said polyhydroxylated fatty alcohols or derivatives thereof are isolated in substantially pure form.

12. The method of claim 2, wherein said polyhydroxylated fatty alcohols or derivatives thereof are synthetically prepared.

13. The method of claim 11, wherein said polyhydroxylated fatty alcohols or derivatives thereof are present at a purity of from about 80% to about 95% w/w of the isolated material.

14. The method of claim 2, wherein said polyhydroxylated fatty alcohols are present in a concentration of from about 0.001% to about 20% w/w of the total composition.

15. (canceled)

16. (canceled)

17. The method of claim 2. wherein said polyhydroxylated fatty alcohols or derivatives thereof or combination thereof are selected from the group consisting of, 1-Acetoxy-2,4-dihydroxy-16-heptadecene or 1,2-dihydroxy-4-acetoxy-16-heptadecene, 1-Acetoxy-2,4-dihydroxy-16-heptadecyne or 1,2-dihydroxy-4-acetoxy-16-heptadecyne

18. (canceled)

19. The method of claim 2, wherein said composition further comprises an additional compound selected from the group consisting of anesthetics; anti-allergenic; antimicrobials; antifungals; anti-inflammatories; antiseptics; chelating agents; colorants; depigmenting agents; emulsifiers; humectants; lubricants; pharmaceutical agents; preservatives; skin penetration enhancers; stabilizers; sunscreens; surfactants; thickeners; and viscosity modifiers.

20. The method of claim 19, wherein said composition is in a form suitable for topical administration.

21. (canceled)

22. (canceled)

23. (canceled)

24. The method of claim 20, wherein said inflammatory disease comprises contact or atopic dermatitis.

25. The method of claim 20, wherein said inflammatory disease comprises psoriasis.

26. The method of claim 20, wherein said inflammatory disease comprises skin inflammatory disorders.

27. (canceled)

28. (canceled)

29. (canceled)

30. The method of claim 2, wherein said composition is in a form suitable for systemic administration.

31. (canceled)

32. The method of claim 30, wherein said inflammatory disease comprises atherosclerosis.

33. The method of claim 30, wherein said inflammatory disease comprises inflammatory bowel disease.

34. The method of claim 30, wherein said inflammatory disease comprises arthritis.

35. (canceled)

36. (canceled)

37. (canceled)

38. (canceled)

39. The method of claim 30, wherein said disease is Crohn's disease.

40-43. (canceled)
